SEQUENCE LISTING (1 sequence)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

(19) United States
(12) Patent Application Publication
(10) Pub. No.: US 2026/0124557 A1
(43) Pub. Date: May 7, 2026

(54) ANTIGEN CHROMATOGRAPHY COLUMNS AND METHODS OF USE THEREOF

(71) Applicant: Waters Technologies Corporation, Milford, MA (US)

(72) Inventors: Nathan Canniff, Concord, MA (US); Kevin Wyndham, Worcester, MA (US)

(73) Assignee: Waters Technologies Corporation, Milford, MA (US)

(21) Appl. No.: 19/371,413

(22) Filed: Oct. 28, 2025

Related U.S. Application Data
(60) Provisional application No. 63/712,652, filed on Oct. 28, 2024.

(51) Int. Cl. B01D 15/20; B01D 15/38; B01D 15/42; C07K 1/22; G01N 33/68

(52) U.S. Cl. CPC B01D 15/206; B01D 15/203; B01D 15/3809; B01D 15/424; C07K 1/22; G01N 33/6854

(57) ABSTRACT

Disclosed herein are antigen chromatography columns. Said antigen chromatography columns utilize streptavidin-functionalized particles, wherein the streptavidin binding sites are occupied with one or more biotinylated antigens. In this way, said antigen chromatography columns may be used to isolate, purify, and characterize antibodies (or other proteins) that specifically bind to said antigens.

### CROSS-REFERENCE TO RELATED APPLICATION

This application claims priority from and the benefit of U.S. Application No. 63/712,652 filed on Oct. 28, 2024. The entire contents of this application are incorporated herein by reference.

### SEQUENCE LISTING

The instant application contains a ST.26 Sequence Listing which has been submitted electronically in XML format and is hereby incorporated by reference in its entirety. Said XML copy, created on Oct. 24, 2025, is named W-4759-US02_ST26 and is 2,180 bytes in size.

### FIELD OF INVENTION

The present disclosure relates generally to methods of generating antigen chromatography columns and methods of use thereof.

### BACKGROUND

The development of new monoclonal antibody therapeutics against a particular target antigen requires several steps, including the generation of potential antibodies and the secondary screening of said antibodies for specific binding. Existing methods for screening antibodies, such as enzyme-linked immunosorbent assays (ELISAs) and surface plasmon resonance (SPR) are time intensive, can have high interexperiment variability, and vary in throughput. Accordingly, there exists a need for new methods of screening antibodies for the ability to specifically bind to a target antigen.

### SUMMARY OF INVENTION

In general, the present technology is directed to antigen chromatography columns for use in the isolation, purification, and characterization of antibodies. In particular, the present technology is directed to chromatography columns comprising nonporous particles conjugated to streptavidin, resulting in a streptavidin column. The streptavidin column effectively serves as an immobilized substrate with the capacity to bind any biotinylated molecule, including biotinylated antigens. Accordingly, the present technology enables the preparation of an antigen chromatography column, thereby allowing for the isolation and purification of antibodies or antigen-binding fragments that specifically bind to said antigen.

Accordingly, in one aspect disclosed herein is a method of loading a biotinylated antigen to a streptavidin column to form an antigen chromatography column, the method comprising selecting a streptavidin column having one or more molecules of streptavidin with accessible binding sites and applying a solution of biotinylated antigens to the streptavidin column such that the biotinylated antigens bind to a portion of the plurality of accessible binding sites. The particles comprise a nonporous core, a hydrophilic surface of an outer layer of the nonporous polymer core and one or more molecules of streptavidin conjugated to the hydrophilic surface and an average particle size between 1-10 μm.

In some embodiments, the biotinylated antigens are applied to the streptavidin column using a high-performance liquid chromatography (HPLC) system, ultra-high performance liquid chromatography (UHPLC) system, or fast protein liquid chromatography (FPLC) system. In some embodiments, the solution of biotinylated antigens is applied for a time sufficient to bind to at least 50% of the plurality of the accessible binding sites of streptavidin.

In some embodiments, the method comprises a step of washing the streptavidin column prior to applying the solution of biotinylated antigens. In some embodiments, the washing comprises applying a wash solvent via a liquid chromatography system. In some embodiments, the wash solvent comprises acetonitrile. In some embodiments, the wash solvent further comprises phosphoric acid. In some embodiments, the wash solvent comprises a sodium phosphate buffer solution.

In one aspect, provided herein is a method for enriching a target antibody, the method comprising: i) providing the antigen chromatography column resulting from the methods described above, ii) washing the column with a binding buffer, iii) applying a solution containing the target antibody to the antigen chromatography column; and iv) washing the column with an elution buffer such that the target antibody is eluted from the column.

In one aspect, provided herein is a method for enriching a target protein, the method comprising: i) providing the antigen chromatography column resulting from the methods described above, ii) washing the column with a binding buffer, iii) applying a solution containing the target protein to the antigen chromatography column; and iv) washing the column with an elution buffer such that the target protein is eluted from the column.

In some embodiments, the antigen chromatography column is connected to a high-performance liquid chromatography (HPLC) system, ultra-high performance liquid chromatography (UHPLC) system, or fast protein liquid chromatography (FPLC) system. In some embodiments, the elution buffer is at least 3 orders of magnitude more acidic than the binding buffer. In some embodiments, the binding buffer has a pH between 7.4-8.0. In some embodiments, the elution buffer has a pH between 1.3-2.3. In some embodiments, the target antibody or protein elutes from the antigen chromatography column with a peak volume of at least two times more than that of the target antibody or protein eluted from a conventional column comprising a plurality of porous particles.

In some embodiments, the target antibody or protein elutes from the antigen chromatography column in less than 5 minutes.

In some embodiments, the method further comprises a step of detecting the eluting target antibody or protein with an ultraviolet spectroscopy detector, a fluorescence spectroscopy detector, and/or a mass spectrometry detector.

In some embodiments, the method further comprises a step of washing the antigen chromatography column with an equilibration buffer or the binding buffer. In some embodiments, said steps are repeated to allow for re-use of the column.

In some embodiments, the nonporous polymer core comprises divinylbenzene, such as divinylbenzene (80). In some embodiments, the coated hydrophilic surface is selected from the group consisting of: (3-glycidyloxypropyl) trimethoxysilane, (3-glycidyloxypropyl)triethoxysilane, glycidol, glyceroltriglycidyl ether, polyacrylate, and poly(methyl acrylate). In some embodiments, at least a portion of an interior surface of the antigen chromatography column body is coated with alkylsilyl material. In some embodiments, the alkylsilyl material is a hydrophilic, non-ionic layer of polyethylene glycol silane.

In some embodiments, the biotinylated antigen is an extracellular protein, a membrane protein, or an intracellular protein. In some embodiments, the antigen is expressed on the surface of a cell, such as an immune cell or a cancer cell. In some embodiments, the antigen is selected from the group comprising PD-1, PD-L1, PDL2, angiopoietin, BCMA, CD19, CD20, CD22, CD25, CD30, CD33, CD37, CD38, CD52, CD56, CD123, LAG-3, cMet, DLL, EpCAM, FGF, GD2, HER2, mesothelin, nectin-4, PDGFRalpha, RANKL, SLAMF7, TROP2, VEGF (e.g., VEGF-A), VEGF-R, Claudin 18.2, DKK1, CD3, CD4, CTLA-4, clusterin, tau, alpha-synuclein, CNX, MHC, Galectin-9, B7-H5 Vista, spike protein, TNF-α, TIGIT, FcRN and tumor associated antigens (TAA, e.g., PSMA, meothelin, EpCAM, HER2, EGFR, etc.). In some embodiments, the antigen is a shell protein present in an adeno-associated virus (AAV), such as VP1, VP2, or VP3. In some embodiments, the solution of biotinylated antigens comprises a first biotinylated antigen and a second biotinylated antigen. In some embodiments, the antigen comprises a ubiquitin binding domain. In some embodiments, the antigen is a protein that specifically binds to an IgE antibody.

In any of the above embodiments, the biotinylated antigen is not a biotinylated antibody or antigen-binding fragment thereof.

### BRIEF DESCRIPTION OF THE DRAWINGS

The technology will be more fully understood from the following detailed description taken in conjunction with the accompanying drawings, in which:

FIG. 1A and FIG. 1B shows the nonporous particle functionalized with streptavidin and the antigen chromatography workflow. FIG. 1A provides a representative example of the functionalized particle. FIG. 1B shows the workflow for generating the antigen chromatography column.

FIG. 2 demonstrates the ability for a biotinylated PD-1 antigen to bind to a column containing streptavidin-functionalized particles.

FIG. 3 demonstrates the results of using a PD-1 antigen chromatography column to isolate and purify pembrolizumab.

FIG. 4A demonstrates streptavidin leaching from a column of the present technology after a first wash method.

FIG. 4B demonstrates streptavidin leaching from a column of the present technology after an alternative wash method.

FIG. 5A, FIG. 5B and FIG. 5C show the results of using a PD-1 antigen chromatography column to isolate and purify PD-L1. FIG. 5A shows the breakthrough from binding. FIG. 5B shows the elution with 120 mM HCl. FIG. 5C shows the elution with 240 mM phosphoric acid.

FIG. 6A, FIGS. 6B and 6C show the results of using a PD-1 antigen chromatography column to isolate and purify PD-L1. FIG. 6A shows the breakthrough from binding. FIG. 6B shows the elution with 120 mM HCl. FIG. 6C shows the elution with 240 mM phosphoric acid.

### DETAILED DESCRIPTION

Disclosed herein are methods for preparing antigen chromatography columns and uses thereof. In order that the technology may be more readily understood, certain terms are first defined. In addition, it should be noted that whenever a value or range of values of a parameter are recited, it is intended that values and ranges intermediate to the recited values are also part of this disclosure. The word “about” if not otherwise defined means±5%. It is also to be noted that as used herein and in the claims, the singular forms “a,” “an” and “the” include plural references unless the context clearly dictates otherwise.

### Definitions

As used herein, the term “antigen chromatography column” refers to a chromatography column comprising particles conjugated to streptavidin molecules, wherein one or more binding sites of the streptavidin molecules are bound to a biotinylated antigen. The antigen chromatography columns of the present technology may be used to identify, isolate, and/or purify target molecules that bind to said antigen. In some embodiments, the antigen chromatography columns of the present technology may be used to identify, isolate, and/or purify target antibodies that specifically bind to said antigen (e.g, antigen-antibody interactions). In some embodiments, the antigen chromatography columns of the present technology may be used to identify, isolate, and/or purify target proteins that bind to said antigen (e.g, protein-protein interactions).

As used herein, the term “conjugate” refers to a compound formed by the chemical bonding of a reactive functional group of one molecule, such as streptavidin, with an appropriately reactive functional group of another molecule, such as an epoxide. An example of suitable reactive functional groups is a nucleophile/electrophile pair. For instance, the nucleophile may be an amine group from an amino acid of streptavidin, and the electrophile is an epoxide.

As used herein, the term “conjugated” refers to the linkage of two molecules formed by the chemical bonding of a reactive functional group of one molecule, such as streptavidin, with an appropriately reactive functional group of another molecule, such as an epoxide.

As used herein, the term “functionalized” refers to a particle that comprises one or more antigen conjugated to its surface. In some embodiments, the antigen is a protein or a fragment of said protein, however the antigen is not an antibody or antigen-binding fragment thereof.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)₂, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)₂ fragments refer to antibody fragments that lack the Fc portion of an intact antibody.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, a camelid, an affibody, a nanobody, an aptamer, or a domain antibody.

As used herein, the term “bispecific antibody” refers to an antibody that is capable of binding at least two different antigens.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

The term “porous” as used herein, refers to a material that has a pore volume that is greater than 0.15 cc/g. Preferably, porous particles have a pore volume that is 0.15 cc/g or greater (e.g., 0.2 cc/g). Pore volume is determined using methods known in the art based on multipoint nitrogen sorption experiments (Micromeritics ASAP 2400; Micromeritics Instruments Inc., Norcross, GA).

The term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Unless defined otherwise, all technical and scientific terms used herein have the same meaning as commonly understood by one of ordinary skill in the art to which this disclosure belongs.

### Particles for Use in Preparing the Streptavidin Column

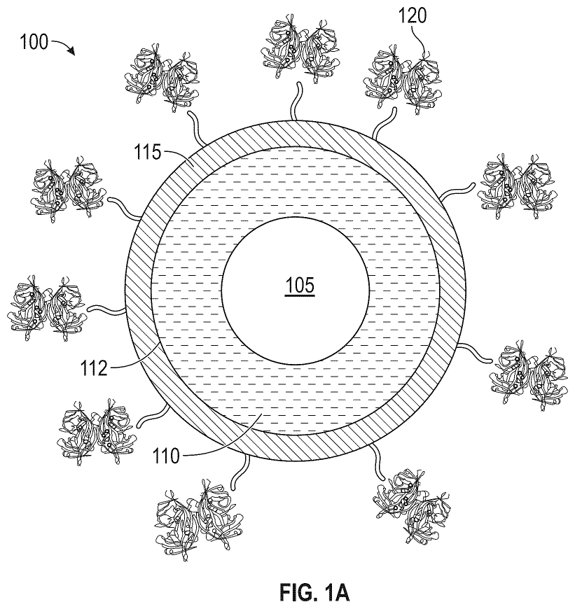

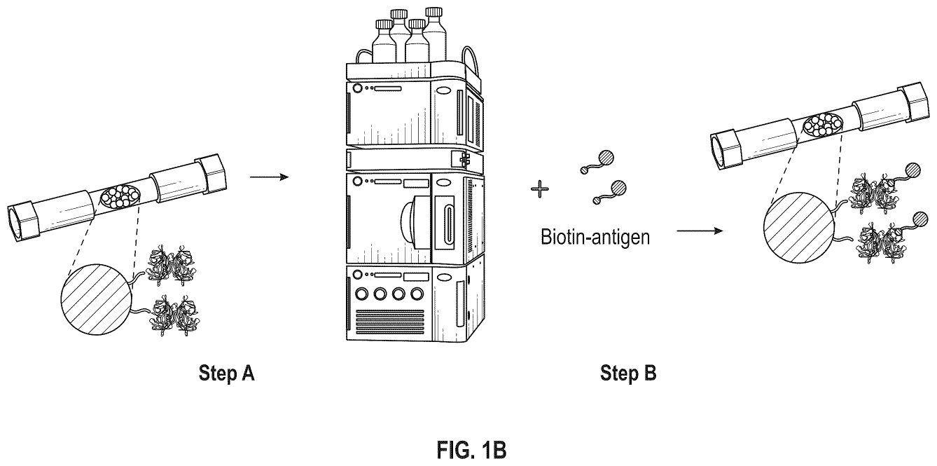

FIG. 1A illustrates an embodiment of a streptavidin-functionalize particle having a nonporous polymer core in accordance with the present technology. The particle illustrated in FIG. 1A has a form factor (e.g., highly spherical, nonporous, and rigid) to withstand operating conditions of HPLC and UHPLC. Particle 100 includes a nonporous polymer core 112 having an inner core region 105 and a radially extending region 110 surrounding the inner core region 105. The inner core region 105 typically is formed of a polymer or a homogenous blend of polymers, whereas the radially extending region 110 typically is formed of two or more polymers to form a gradient within this region. For example, core region 105 can be formed of polystyrene, whereas radially extending region 110 contains a gradient composition transitioning from 100% polystyrene to 80% to 100% DVB with any remainder being polystyrene. In some embodiments, the nonporous particle is highly spherical with a smooth surface. In some embodiments, the nonporous particle is highly spherical with a bumpy, convex surface.

In one embodiment, to form nonporous polymer cores 112, the following three steps were used. In step one: 561.1 g of reagent alcohol (90% ethanol, ^{˜}5% methanol and ^{˜}5% isopropanol), 16.9 g of polyvinylpyrrolidone (PVP-40, average molecular weight 40,000), 1.6 g of 2,2′-Azobis(2-methylpropionitrile) (AIBN), 6.7 g of Triton™ N-57, 80.1 g of styrene and 2.4 g of poly(propylene glycol) dimethacrylate (average molecular weight 560) were charged into a reactor. After purging with nitrogen, the reaction mixture was heated to 70° C. with stirring and was held at 70° C. until the completion of all the reaction steps. In step two: after the step one reaction mixture was held at 70° C. for 3 hours, a solution containing 52.0 g of DVB 80, 24.0 g of styrene, 51.0 g of PVP-40, 1080.4 g of reagent alcohol (90% ethanol, ^{˜}5% methanol and ^{˜}5% isopropanol) and 54.1 g of p-xylene was added to the reaction mixture at a constant flow rate over two hours. In step three: after the completion of solution charge in step two, a primer coating solution containing 31.2 g of glycidyl methacrylate (GMA), 6.2 g of ethylene glycol dimethacrylate (EDMA), 12.9 g of PVP-40 and 381.9 g of reagent alcohol (90% ethanol, ˜5% methanol and 5% isopropanol) was added to the reaction mixture at a constant flow rate over 1.5 hours. After the reaction mixture was held at 70° C. for a total of 20 hours, the particles were separated from the reaction slurry by filtration. The particles were then washed with methanol, followed by tetrahydrofuran (THF), and followed by acetone. The final product was dried in vacuum oven at 45° C. overnight. 91.8 g of monodisperse 2.3 μm polymer particles were obtained.

While the embodiment shown in FIG. 1A illustrates that the nonporous polymer core 112 has two regions (the inner core region 105 and the radially extending region 110), that need not be the case. Other embodiments may feature a nonporous polymer core having a singular region, i.e., the nonporous polymer core extends from the center of the particle to an outer surface of the nonporous polymer core 112. Other embodiments of nonporous polymer cores and particles suitable for use with the present technology are described in U.S. Patent Publication No. 2019/0322783.

As illustrated in FIG. 1A, a hydrophilic surface or layer 115 is formed on an outer surface (i.e., opposite to the center region 105) of the nonporous polymer core. In one embodiment, the hydrophilic surface 115 is formed through the application of a hydrophilic primer coating solution containing 36.2 g of glycidyl methacrylate (GMA), 7.44 g of ethylene glycol dimethacrylate (EDMA), 8.21 g of PVP360 (PVP360, average molecular weight 360,000) and 489.4 g of reagent alcohol (90% ethanol, ^{˜}5% methanol and ^{˜}5% isopropanol). This solution was added into to a mixture containing the nonporous polymer cores at a constant flow rate over about 1.5 hours to form hydrophilic surface 115.

The above example is provided for illustration purposes only. Other types of hydrophilic surfaces can be applied. For example the hydrophilic layer may also be formed of (3-glycidyloxypropyl) trimethoxysilane, (3-glycidyloxypropyl)triethoxysilane, polyacrylate, and/or poly(methyl acrylate), glycidol, glyceroltriglycidyl ether, or any other type of hydrophilic material.

To form streptavidin-functionalized particles, a linker is used to conjugate the hydrophilic surface 115 to the streptavidin. Referring to FIG. 1A, particle 100 results from the attachment of streptavidin to the hydrophilic surface through use of the linker. In one embodiment, the streptavidin 120 is attached to the hydrophilic surface 115 by a ring opening of a surface epoxide. That is, an epoxy linker is utilized. That need not be the case. Other linkages can be used. For example, instead of attaching using an epoxy linker, attachment can be made using an amide bond formation, a cyanogen bromide reaction, or aldehyde condensation. One of ordinary skill in the art would understand that a number of linkers are suitable for use with the present technology.

In some embodiments, the epoxy linker has a formula:

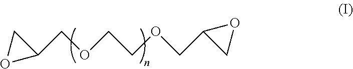

wherein n (the number of ethylene oxide repeating units) is an integer from 1 to 150. In some embodiments, n is 1, 2, 3, 4, 5, 6, 7, 8, 9, 10, 11, or 12. In some embodiments, n is 1, 4, or 9. In some embodiments, n is 1.

### Methods of Making Antigen Chromatography Columns

In one aspect, the nonporous particles are conjugated to one or more molecules of streptavidin. In some embodiments, the streptavidin molecules are present at 1-5 μg/mg of particle. In some embodiments, the streptavidin molecules are present at 1-2, 2-3, 3-4, or 4-5 μg/mg of particle. In some embodiments, the nonporous particles are conjugated to avidin. In some embodiments, the nonporous particles are conjugated to a protein that binds to biotin. One of ordinary skill in the art would understand that the use of avidin or streptavidin would achieve similar results. The nonporous particles conjugated to streptavidin are then suitable for use in preparing antigen chromatography columns with a biotinylated antigen.

FIG. 1B shows the process of preparing the antigen chromatography column, wherein the column is first packed with the streptavidin-conjugated particles to provide the streptavidin column (step a). This is then connected to a liquid chromatography device. The biotinylated antigen is then flowed on the column, wherein the biotinylated antigen binds to one or more accessible streptavidin binding sites (step b).

As used herein, the term “biotinylated antigen” refers to a biotinylated molecule, such as a protein, that is not an antibody or antigen-binding fragment thereof. In some embodiments, the molecule is biotinylated with a biotin derivative, including but not limited to, iminobiotin, desthiobiotin, difulfide biotin azide, disulfide biotin alkyne, or other biotin derivatives.

An advantage of the method of pumping the biotinylated antigen across a bed of particles packed into a device includes precise metering of reagents, contact times, and ability to use post-column detectors (e.g., use of detector to monitor amount of biotinylated molecule eluting from the column versus loading on the column).

The pump system used to pump fluids across the plurality of particles in the chromatographic devices include UHPLC system pumps, HPLC system pumps, and FPLC system pumps. These pump-column systems can be connected to a post-column detector (UV, TUV, PDA, RI, MALS, MS, FL) or they can flow without attachment to a detector. Multiple columns can be coupled in series or in parallel using tubing to increase throughput. The effluent of the columns can be isolated and reused or directed to suitable waste container.

After flowing the solution of biotinylated antigens through the plurality of particles packed in the column, the chromatographic device can be washed with water, PBS, buffer, or storage buffer, and then stopped or enclosed to prevent evaporation and, if desired, stored in a refrigerator until ready for use.

In some embodiments, a column packed with a plurality of functionalized streptavidin particles can be washed with water, a buffer or storage solution, and/or an acetonitrile-based solution (e.g., 20% acetonitrile and 1% phosphoric acid) prior to adding the solution containing biotinylated antigen. The column packed with the plurality of functionalized streptavidin particles can be stored prior to the loading of the biotinylated antigen. That is, between step a and step b of the method shown in FIG. 1B.

In one embodiment, the antigen chromatography column is prepared as follows. The nonporous particles functionalized with streptavidin are packed into a chromatography column, resulting in a streptavidin column. A number of column sizes and materials are suitable for use in the methods disclosed herein. In some embodiments, the column material is stainless steel, polyetheretherketone (PEEK) lined steel, titanium, or a stainless steel alloy such as MP35n. In some embodiments, the column has an internal diameter ranging from 75 μm to 4.6 mm. In some embodiments, the column has a length between 5 to 300 mm. In a preferred embodiment, the column has an internal diameter between 1 to 2 mm and a length between 5 to 20 mm. The column surface can be unmodified or modified to generate a high-performance surface. Chromatography columns suitable for use with the methods disclosed herein are compatible with any standard liquid chromatography system, including high-performance liquid chromatography (HPLC) systems, ultra-high performance liquid chromatography (UHPLC) systems, and fast protein liquid chromatography (FPLC) systems.

In some embodiments, the liquid chromatography system is connected in series to a detector. Detectors suitable for use in the methods disclosed herein include detectors for ultraviolet spectroscopy, fluorescence spectroscopy, and/or mass spectrometry. In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected to one or more of the detectors in series.

In some embodiments, the interior surfaces of the column are treated to reduce non-specific binding and enhance overall efficiency of the liquid chromatography system. In particular, an alkylsilyl coating or other high performance surface is provided to limit or reduce non-specific binding of a sample with walls or interior surfaces of a column body. Without wishing to be bound by theory, it is believed that an alkylsilyl coating covering metal surfaces prevent or minimize contact between fluids passing through the column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. Other embodiments of alkylsilyl coatings suitable for use with the present technology are described in U.S. Patent Publication No. 2019/0086371 (see also U.S. Pat. No. 11,709,155) and U.S. Application Publication No. 2022/0118443.

The streptavidin column, comprising the streptavidin-functionalized particles, is then connected to a suitable liquid chromatography system. The biotinylated antigens are flowed through the column, permitting the ionic bonding of the biotin group to one of the accessible streptavidin binding sites. Streptavidin naturally occurs as a homo-tetramer with four available binding pockets for biotin. Due to the stochastic nature of how streptavidin is conjugated to the nonporous particle, a given streptavidin molecule may have 0, 1, 2, 3, or 4 accessible binding sites. In some embodiments, the biotinylated antigen may bind to 0, 1, 2, 3, or 4 accessible binding sites of a given streptavidin molecule. In some embodiments, 50-55%, 55-60%, 60-65%, 65-70%, 70-75%, or more than 75% of the plurality of the accessible streptavidin binding sites are occupied by a biotinylated antigen. The extremely strong affinity between biotin and streptavidin ensures that the biotinylated antigens are immobilized onto the solid phase of the chromatography column. The interaction between biotin and streptavidin is resistant to organic solvents, changes in pH, changes in temperature, detergents, and many concentrations of denaturants. As such, the affinity chromatographic columns disclosed herein are suitable for use with a range of organic solvents, pH, temperatures, and samples.

To increase the selectivity of the antigen chromatography column, it is desirable to maximize the percentage of accessible binding sites of the streptavidin molecules present in the streptavidin column that are bound with a biotinylated antigen. The percentage of accessible binding sites that are occupied, or the extent of saturation, can be determined by monitoring the effluent while preparing the affinity chromatographic column. When initially applying the biotinylated antigen to the streptavidin column, the biotinylated antigen will bind to any accessible streptavidin site. As such, upon initial application of biotinylated antigen, the biotinylated antigen thereof should be present at low levels in the effluent as measured by a detector. In some embodiments, the detector used to monitor the effluent is an ultraviolet detector or a fluorescence detector. As the streptavidin column increases in saturation, the amount of biotinylated antigen will increase in the effluent. The increase in concentration of the biotinylated antigen thereof indicates an increase in the saturation of accessible binding sites of the streptavidin column. If the effluent is monitored over time, the increase in biotinylated antigen present in the effluent will plateau, indicating saturation of the streptavidin column.

In some embodiments, it may be advantageous to endcap the plurality of particles with excess biotin. This process, referred to herein as ‘biotin endcapping’ involves the addition of free biotin to the plurality of particles after a biotinylated antigen is added. The free biotin can interact with remaining, unoccupied binding sites on the streptavidin molecules, increasing efficiency and reducing noise. Said unoccupied streptavidin binding sites may be present due to, for example, incomplete saturation of the column or due to steric hindrance that blocks the biotin of the affinity group from binding to streptavidin.

Below a number of examples are presented including Example 1 which provides a description of making particles for use in a streptavidin column in accordance with the present technology and Example 2 which provides a description of preparing a streptavidin column in accordance with the present technology.

Example 3 provides a method of loading a streptavidin column with a biotinylated antigen that is PD-1. As the biotinylated antigen is loaded onto the streptavidin column, the effluent is monitored using a UV detector.

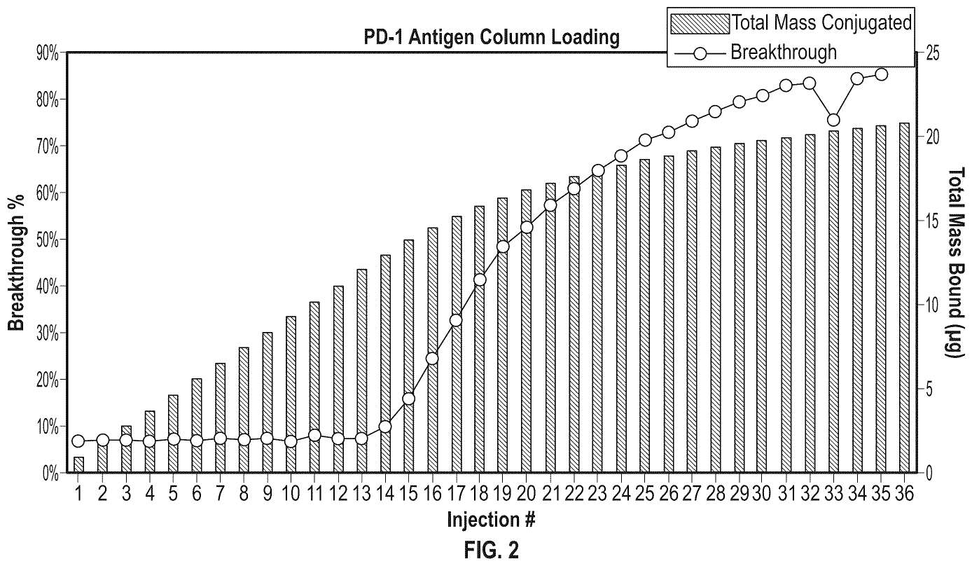

FIG. 2 plots the amount of biotinylated antigen from Example 3 present in the effluent over the duration of the column loading. After ˜14 injections, the percentage of biotinylated antigen present in the effluent increases, indicating the streptavidin column is beginning to reach saturation. After ˜35 injections, the increase in the biotinylated antigen present in the effluent plateaus, meaning that the plurality of accessible binding sites of streptavidin in the column is saturated.

It is expected that a portion of the biotinylated antigen will be present in the effluent during loading due to breakthrough resulting from the stochastic binding process. In some embodiments, approximately 0-10% of the biotinylated antigen will be present in the effluent prior to saturation. In some embodiments, approximately 0-2%, 2-4%, 4-6%, 6-8%, or 8-10% of the biotinylated antigen will be present in the effluent prior to saturation.

The resultant column, wherein a plurality of the accessible binding sites of the streptavidin molecules are bound to a biotinylated antigen, can subsequently be used as an antigen chromatography column-which may be used to isolate and purify one or more antibodies that bind to said antigen. Additionally or alternatively, the antigen chromatography columns provided herein may be used to isolate and purify one or more proteins that bind to said antigen.

### Antigens Suitable for Use in Preparing Antigen Chromatography Columns

A number of antigens are suitable for use in the disclosed technology, provided that the antigens are biotinylated. In some embodiments, the biotinylated antigen is a protein or a fragment of said protein.

In some embodiments, the biotinylated antigen is a protein expressed on the surface of a cell. In some embodiments, the cell is a cancer cell. In some embodiments, the cell is an immune cell. In some embodiments, the immune cell is a monocyte, a macrophage, a granulocyte, a natural killer cell, or a dendritic cell.

In some embodiments, the biotinylated antigen is an extracellular protein.

In some embodiments, the biotinylated antigen is an intracellular protein.

In some embodiments, the biotinylated antigen is selected from the group comprising: PD-1, PD-L1, PDL2, angiopoietin, BCMA, CD19, CD20, CD22, CD25, CD30, CD33, CD37, CD38, CD52, CD56, CD123, LAG-3, cMet, DLL, EpCAM, FGF, GD2, HER2, mesothelin, nectin-4, PDGFRalpha, RANKL, SLAMF7, TROP2, VEGF (e.g., VEGF-A), VEGF-R, Claudin 18.2, DKK1, CD3, CD4, CTLA-4, clusterin, tau, alpha-synuclein, CNX, MHC, Galectin-9, B7-H5 Vista, spike protein, TNF-α, TIGIT, FcRN, and tumor associated antigens (TAA, e.g., PSMA, meothelin, EpCAM, HER2, EGFR, etc.).

In some embodiments, the biotinylated antigen is a shell protein present in an adeno-associated virus. In some embodiments, the shell protein is VP1, VP2, or VP3.

In some embodiments, the biotinylated antigen is a protein having a ubiquitin binding domain. In some embodiments, the biotinylated antigen comprises 1, 2, 3, 4, 5, 6, or more ubiquitin binding domains. Ubiquitin binding domains are known in the art, and include UBA, CUE, GAT, UEV, Ubc, UIM, DUIM, MIU, NZF, A20 Znf, and Znf UBP. Examples of ubiquitin binding domains are further described in Hurley et al., “Ubiquitin-binding Domains” Biochem J. 2006 399:361-372.

In some embodiments, the biotinylated antigen is a protein that binds to an IgE antibody, i.e., an allergen. In some embodiments, the allergen is a protein derived from a food, such as celery, corn, eggs, fruit, legumes, milk, seafood, sesame, soy, tree nuts, wheat. In some embodiments, the allergen is a protein derived from plant pollen, such as grass, weeds, or trees. In some embodiments, the allergen is amerchol L-101, ammonium persulfate, Peru balsam, benzisothiazolinone, benzocaine, benzyl alcohol, benzyl salicylate, 2-bromo-2-nitropropane-1,3,-diol, 4-tert-butylphenolformaldehyde, bacitracin, budesonide, quaternium-15, chloroxylenol, cinnamal, cobalt (II) chloride, cocamide, colophonium, clobetasol-17-propionate, toluene-2,5-diamine, 1,3-diphenylguanidine, diazolidinyl urea, dmdm hydantoin, methyldibromo glutaronitrile, decyl glucoside, bisphenol A, ethyl acrylate, ethylenediamine dihydrochloride, 2-hydroxyethyl methacrylate, benzophenone-4, linalool, limonene, imidazolidinyl urea, n-isopropyl-n-phenyl-4-phenyleneidamine, iodopropynyl butylcarbamate, lidocaine, hydroxyisohexyl 3-cyclohexene carboxaldehyde, lauryl polyglucose, 2-mercaptobenzothiazole, methyl methacrylate, thiuram, paraben, black rubber, neomycin sulfate, nickel (II) sulfate hexahydrate, 2-n-octyl-4-isothiazolin-3-one, p-phenyleneidamine, potassium dichromate, propyl gallate, propolis, polymyxin B, pramoxine, sodium benzoate, sorbitan oleate, sorbitan sesequioleate, sodium metabisulfite, toluenesulfonamide formaldehyde resin, tixocortol-21-pivalate, tea tree oil, tocopherol, lanolin alcohol, Ylang ylang oil, amidoamine, benzalkonium, chlorohexidine digluconate, methylisothiazolinone, cocamidopropyl betaine, 3-(dimethylamino)-1-propylamine, formaldehyde, methylisothiazolinone, oleamidopropyl dimethylamine, propylene glycol. In some embodiments, the allergen is a protein derived from peanuts.

As used herein and throughout, an antigen may be an intact, full-length protein or a fragment of said protein. For example, a fragment of said protein may comprise up to 10%, 20%, 30%, 40%, 50%, 60%, 70%, 80%, 90%, 95%, or 99% of the full-length protein sequence as determined by the amino acid sequence.

For example, but not by way of limitation, human PD-1 has an amino acid sequence identified by UniProt accession number Q15116. PD-1 has the amino acid sequence of:

(SEQ ID: 1)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

Thus, a biotinylated antigen of PD-1 may comprise the full-length amino acid sequence as set forth above, or may comprise 10%, 20%, 30%, 40%, 50%, 60%, 70%, 80%, 90%, 95%, or 99% of the full-length amino acid sequence of PD-1.

### Methods of Using Antigen Chromatography Columns

The resultant antigen chromatography columns of the disclosed technology can be used to identify antibodies or antigen-binding fragments thereof that bind to the antigen or antigens present in the antigen chromatography column. Additionally or alternatively, the resultant antigen chromatography columns of the disclosed technology can be used to identify proteins or fragments of said proteins that bind to the antigen or antigens present in the antigen chromatography column. That is, the present technology allows for the isolation and purification of both antibody-antigen interactions and non-antibody protein-antigen interactions.

The antigen chromatography column is suitable for use in conjunction with any liquid chromatography system as described above. In some embodiments, the liquid chromatography system is an HPLC, UHPLC, or FPLC.

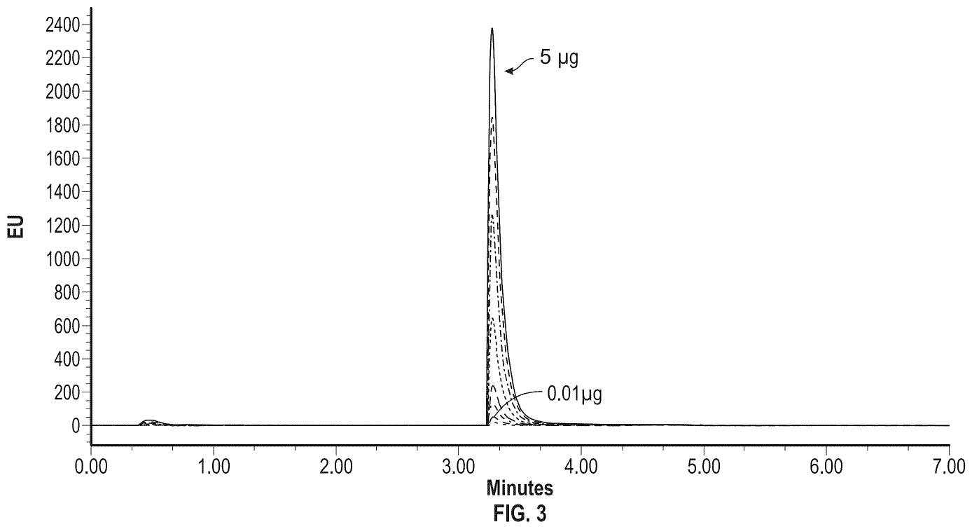

Example 4 describes a method of using an antigen chromatography column, wherein the antigen is PD-1. Said antigen chromatography column can be used to capture antibodies that specifically bind to PD-1, such as pembrolizumab. FIG. 3 demonstrates that the antigen chromatography column may be used to bind and elute pembrolizumab, an antibody known to specifically bind to PD-1.

Example 5 describes a first method of evaluating streptavidin leaching from a column.

Example 6 describes a second method of evaluating streptavidin leaching from a column.

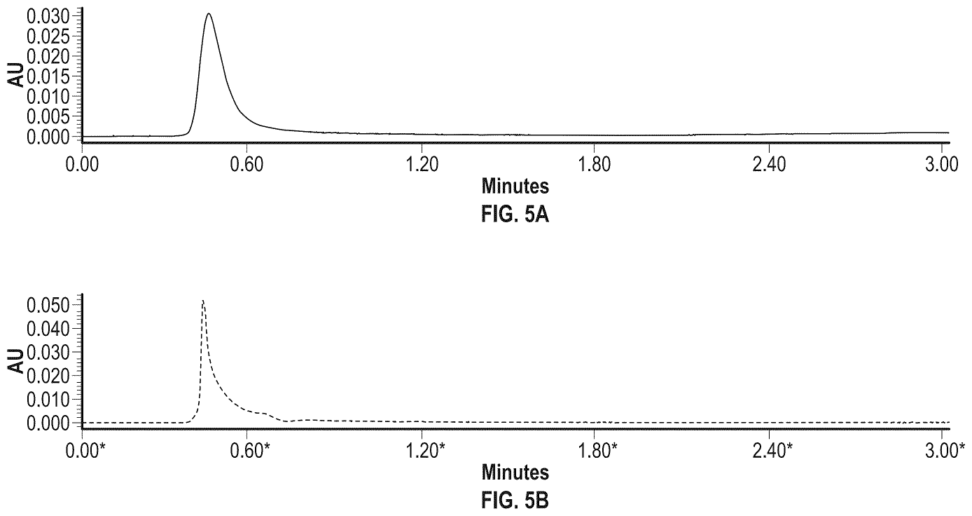

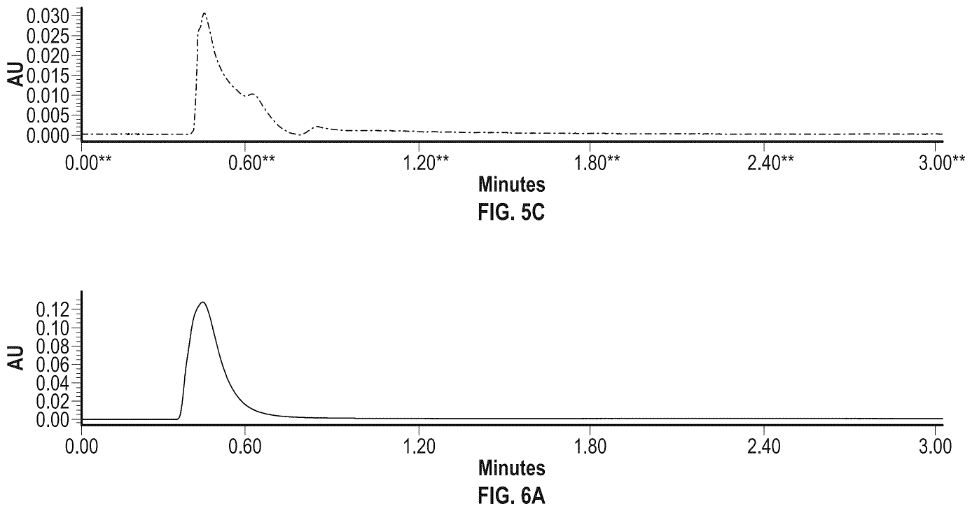

Example 7 describes a method of using an antigen chromatography column, wherein the antigen is PD-1. Said antigen chromatography column can be used to capture antibodies that specifically bind to PD-1, such as PD-L1. FIGS. 5A, 5B, and 5C demonstrates that the antigen chromatography column may be used to bind and elute PD-L1, an antibody known to specifically bind to PD-1.

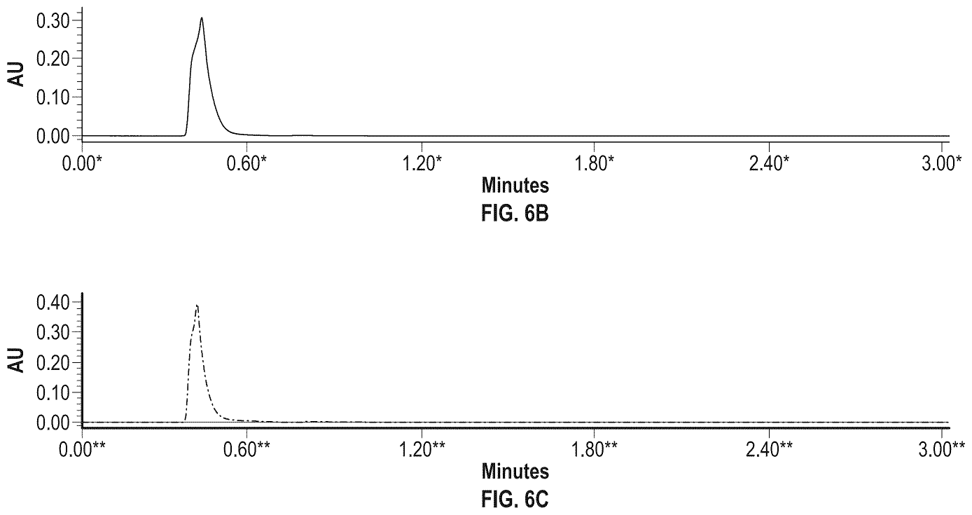

Example 8 describes a method of using an antigen chromatography column, wherein the antigen is PD-1. Said antigen chromatography column can be used to capture antibodies that specifically bind to PD-1, such as PD-L2. FIGS. 6A, 6B, and 6C demonstrates that the antigen chromatography column may be used to bind and elute PD-L2, an antibody known to specifically bind to PD-1.

Example 9 describes a method of preparing and using an antigen chromatography column, wherein the antigen is a protein or fragment thereof present on the surface of a cell or particle, such as a lipid nanoparticle.

Example 10 describes a method of preparing and using an antigen chromatography column having a first and second antigen present in the column.

Example 11 describes a method of preparing and using an antigen chromatography column, wherein the antigen comprises one or more ubiquitin binding domains.

Example 12 describes a method of preparing and using an antigen chromatography column wherein the antigen is a biomarker associated with a disease or disorder, such as an autoimmune disorder, a neurodegenerative disorder, or hematologic disorder. Additionally or alternatively, the biomarker may be a protein that specifically binds to an IgE antibody.

Buffers suitable for use in liquid chromatography are well known in the art. It is understood that a range of binding buffers are suitable for use with the disclosed technology and a person of ordinary skill in the art could determine without undue experimentation the appropriate binding buffer that is suitable with a given target antibody or target protein. In one aspect, the binding buffer has a pH between 7.0 and 8.0. In some embodiments, the pH of the binding buffer is between 7.0-7.1, 7.1-7.2, 7.2-7.3, 7.3-7.4, 7.4-7.5, 7.5-7.6, 7.6-7.7, 7.7-7.8, 7.8-7.9, or 7.9-8.0. In one embodiment, the pH of the binding buffer is 7.3. In one embodiment, the pH of the binding buffer is 7.4. In one aspect, the elution buffer has a pH between 1.0 and 3.0. In some embodiments, the pH of the elution buffer is between 1.0-1.2, 1.2-1.4, 1.4-1.6, 1.6-1.8, 1.8-2.0, 2.0-2.2, 2.2-2.4, 2.4-2.6, 2.6-2.8, or 2.8-3.0. In one embodiment, the pH of the elution buffer is 1.3. In one embodiment, the pH of the elution buffer is 2.3. It is important that the pH of the elution buffer be at least 3 magnitudes more acidic than the binding buffer.

In some embodiments, the target antibody or target protein is eluted from the column using a step elution, wherein the mobile phase is switched from the binding buffer to the elution buffer. In some embodiments, the target antibody or target protein is eluted from the column using a gradient elution, wherein the binding buffer is transitioned to the elution buffer as a gradient.

In one aspect, the antigen chromatography columns of the present technology can be re-used for subsequent assays. In this regard, after the elution of the target antibody or target protein from the column, the column is then re-equilibrated. Re-equilibration involves flowing buffer through the column such that any residual material from the previous assay is removed. In some embodiments, re-equilibration is performed with the binding buffer. In some embodiments, the re-equilibration is performed with an equilibration buffer. The equilibration buffer may have a pH that is equal to or lower than the binding buffer. In one aspect, the present technology allows for rapid re-equilibration of the antigen chromatography column. In some embodiments, the antigen chromatography column is re-equilibrated for 1-10 minutes, 1-2, 2-3, 3-4, 4-5, 5-6, 6-7, 7-8, 8-9, or 9-10 minutes. In some embodiments, it may be required that the column is re-equilibrated for longer periods of time.

Following re-equilibration, the antigen chromatography column is ready to be re-used for antibody capture assays or protein-protein interaction assays. Antigen chromatography columns of the instant technology can be re-used 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, or 200 or more times without degradation or noticeable loss of specificity. In one aspect, the antigen chromatography column of the instant technology can be stored in-between uses. Buffers suitable for the storage of affinity chromatographic columns are well known in the art.

### EXAMPLES

### Example 1: Addition of an Epoxy Linker to Hydrophilic Particles

The nonporous, epoxy-modified hydrophilic particles for use in the disclosed methods were prepared as follows. As a first step, 1500 g of reagent alcohol (90% ethanol, ˜5% methanol, and ˜5% isopropanol), 45.1 g of polyvinylpyrrolidone (PVP-40), 4.8 g of 2,2′-Azobis(2-methylpropionitrile), 5.9 g of Triton™ N-57, and 81.7 g of styrene were charged into a reactor. After the reactor was purged with nitrogen gas, the reaction mixture was heated to and maintained at 70° C. with stirring for 3 hours.

After three hours, a solution containing 110.4 g of divinylbenzene 80 (DVB), 39.7 g of PVP-40, 510 g of reagent alcohol, and 100.2 g of p-xylene was added to the reaction mixture at a constant flow rate over two hours. Following this step, a primer coating solution containing 26.0 g of glycidyl methacrylate (GMA), 26.0 g of ethylene glycol dimethacrylate (EDMA), 36.4 g of PVP-40, and 560 g of reagent alcohol were added to the reaction mixture at a constant flow rate over 1.5 hours.

The reaction mixture was maintained at 70° C. for a total of 20 hours, after which the particles were separated from the reaction slurry by filtration. The particles were then washed sequentially with methanol, tetrahydrofuran (THF), and acetone. The final product was dried in a vacuum oven at 45° C., resulting in monodisperse 3.5 μm polymer particles. These particles contain a gradient polystyrene/DVB core with a poly(GMA/EDMA) primer (FIG. 1A). While the above reaction conditions generate 3.5 μm polymer particles, it is understood that particles ranging in sizes from 1.0 μm to 10 μm (e.g., 1.5 μm to 8 μm, such as 3.5 μm) are within the scope of the disclosure. By altering the concentrations of PVP-40, 2′2-Azobis(2-methylpropionitrile), and Triton N-57, one of ordinary skill in the art could generate a range of particle sizes.

The resultant 3.5 μm, polystyrene/DVB particles with the poly(GMA/EDMA) primer were then coated with a hydrophilic layer. 70 g of the particles were hydrolyzed in 0.5M H₂SO₄ at 60° C. for 1-20 hours. The hydrolyzed particles were washed sequentially with MilliQ water and methanol, and then dried under vacuum at 45° C. overnight. The dried particles were added into a 1 L three-necked round bottom flask with an overhead stirring motor, stirring shaft, and stir blade, a water cooled condenser, a nitrogen inlet, and a probe-controlled heating mantle. 700 mL of anhydrous diglyme (diethylene glycol dimethyl ether) was added, the flask sealed, and purged with nitrogen for 15 minutes with moderate stirring. 2.0 g of potassium tert-butoxide was added, and the reaction was raised to 70° C. To generate the hydrophilic layer, a mixture of 10.5 g glycidol, 2.6 g of glyceroltriglycidyl ether, and 14.9 g of anhydrous diglyme was prepared separately and added to the particle mixture in four equal aliquots in 30 minute intervals. The reaction was held at 70° C. for 20 hours, cooled to RT, and filtered. The resulting particles were washed sequentially with water 6 times, methanol 3 times, and then dried under vacuum overnight at 45° C. The following procedure results in a hydrophilic layer that is 2-4% (by weight) of the entire particle (FIG. 1A).

20 g of the resultant 3.5 μm particles with the hydrophilic coating were added to a mixture of 100 g of ethylene glycol diglycidyl ether (EGDGE) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable functionalization of the particle surface (FIG. 1A).

Alternatively, 20 g of the resultant 3.5 μm particles with the hydrophilic coating were added to a mixture of 100 g of poly(ethylene glycol) diglycidyl ether (a compound of Formula 1, wherein n is 4, also known as PEGDE 200) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable 3.3 μg streptavidin coverage per mg particle.

Alternatively, 20 g of the resultant 3.5 μm particles with the hydrophilic coating were added to a mixture of 100 g of poly(ethylene glycol) diglycidyl ether (a compound of Formula 1, wherein n is 9, also known as PEGDE 400) and 100 g of MeOH at room temperature. 1 mL of 50% sodium hydroxide in water was added and the reaction was stirred continuously for 20 h. The particles were isolated by filtration, washed with 40 mL of MeOH ten times, and partially dried under nitrogen flow. The particles were stored for later use in a methanol wet bed at 4° C. The resultant particles have sufficient epoxide content to enable 3.4 μg streptavidin coverage per mg particle.

### Example 2: Preparation of a Streptavidin Column

Particles were prepared as described in Example 1 and functionalized with streptavidin. 1.5 g of particles were mixed in 7 mL of a 50-100 mM buffer (pH 8-9.2). To this, 1.5 mL of a 10 mg/mL solution of streptavidin (15 mg) was added. Next, 21.4 mL of a buffer containing a salting out agent was added dropwise. The reaction was then stirred for 20 hours between 24-37° C. The buffer system, salting out agent and its concentration together I temperature of the reaction can be adjusted to manipulate the extent of streptavidin coverage on a given particle, as shown in Table 1.

Following the 20 h incubation, 1 g of ethanolamine in 4 mL of a buffer solution (e.g., sodium phosphate) was added and the reaction stirred at RT for 3 hours. Particles were then isolated by filtration and washed. The washing process comprises: step 1: three times pH4 water (i.e., adjusted with HCl); step 2: three times with water or water/organic solvent mixture as described in Table 1; step 3: three times with water; and step 4: twice with storage buffer (100 mM PBS, PH 7.3, 0.02% sodium azide). The particles were stored in a sealed container as a slurry in storage buffer (˜10 mL buffer/g of particle) at 4° C. Streptavidin coverage of the particles was determined using a standard bicinchoninic acid assay (BCA). Maximum binding capacity was estimated using the ratio of the molecular weight of streptavidin and a biotinylated antibody multiplied by the binding valency of streptavidin (4) as shown in Table 1.

TABLE 1
Particles Functionalized with Streptavidin

| | | | | | | Maximum |
|---|---|---|---|---|---|---|
| | | | | | Streptavidin | Binding |
| | | Salting | | Particle | Coverage | Capacity |
| Product | Buffer | Out | | Wash 2^{nd} | (μg/mg | (μg IgG/mg |
| # | System | Agent | Temp | Step | particle) | particle) |
| 2a | Phosphate | 1.7M | 24° C. | Water | 2.2 | 24 |
| | Buffer | Ammonium | | | | |
| | (pH 8) | Sulfate | | | | |
| 2b | Phosphate | 2.85M | 24° C. | Water | 3.3 | 36 |
| | Buffer | Ammonium | | | | |
| | (pH 8) | Sulfate | | | | |
| 2c | Phosphate | 2.85M | 37° C. | Water | 4.6 | 50 |
| | Buffer | Ammonium | | | | |
| | (pH 8) | Sulfate | | | | |
| 2d | Phosphate | 2.85M | 37° C. | Acetonitrile/ | 5.2 | 57 |
| | Buffer | Ammonium | | Water | | |
| | (pH 8) | Sulfate | | (1/3) | | |
| 2e | Phosphate | 2.85M | 37° C. | Dimethyl | 4.6 | 50 |
| | Buffer | Ammonium | | sulfoxide/ | | |
| | (pH 8) | Sulfate | | Water (1/9) | | |
| 2f | Carbonate- | 1.5M | 37° C. | Water | 3.9 | 43 |
| | bicarbonate | Sodium | | | | |
| | Buffer | Sulfate | | | | |
| | (pH 9.2) | | | | | |
| 2g | Carbonate- | 1.5M | 37° C. | Dimethyl | 4.0 | 44 |
| | bicarbonate | Sodium | | sulfoxide/ | | |
| | Buffer | Sulfate | | Water (1/9) | | |
| | (pH 9.2) | | | | | |
| 2h | Carbonate- | 1.5M | 24° C. | Dimethyl | 3.4 | 37 |
| | bicarbonate | Sodium | | sulfoxide/ | | |
| | Buffer | Sulfate | | Water (1/9) | | |
| | (pH 9.2) | | | | | |

### Example 3: Preparation of a PD-1 Antigen Chromatography Column

Particles prepared as described in Example 2 using 2.85M ammonium sulfate at 24° C. (product #2b) were packed in a 2.1×20 mm column hardware and stored in storage buffer (100 mM PBS, pH 7.3, 0.02% azide; at ˜10 mL/g particle). The column was stored at 4° C. until ready for use. The column was connected to a liquid chromatography instrument and purged with 100 mM sodium phosphate buffer (pH 7.4) at 0.1 mL/min. Next, a 5 μL injection of a 0.2 mg/mL solution of biotinylated PD-1 was injected onto the column and flowed at 0.1 mL/min for 2 minutes. These injections were repeated and the effluent monitored over 36 injections using a UV detector (280 nm). The UV detector allows for the measurement of the percentage of biotinylated PD-1 eluted.

It was shown that the biotinylated PD-1 was binding to the streptavidin-functionalized beads of the column as indicated by the low level of PD-1 eluting from the column during the initial injections. After ˜13 injections, the amount of biotinylated PD-1 in the effluent increased, indicating that excess biotinylated antigen was not binding to the column and that the streptavidin sites were saturated (FIG. 2). Based on these results, it was estimated that ˜20 μg of the biotinylated PD-1 was bound to the column device. The column was washed with phosphate buffer and then storage buffer.

### Example 4: Binding of Pembrolizumab to a PD-1 Antigen Chromatography Column

The column of Example 3 was used to perform affinity capture of pembrolizumab, an exemplary antibody which binds to the PD-1 antigen. The column was connected to a liquid chromatography instrument and equilibrated with a 100 mM sodium phosphate buffer (pH 7.4) for 5-7 minutes at 1 mL min flow rate.

Once equilibrated, a 5 μL injection of 0.01 mg/mL pembrolizumab was injected onto the column and flowed for 2 minutes at 0.1 mL/min and 1 minute at 1 mL/min to allow for binding. The column effluent was monitored via a fluorescence detector (280 nm excitation, 350 nm emission). After the binding phase, the elution buffer (12 mM HCl) was flowed onto the column at 1 mL/min for 2 minutes. Following this, the column was re-equilibrated with 100 mM sodium phosphate buffer (pH 7.4) for 2 minutes at a 1 mL/min flow rate.

After re-equilibrating the column, a 5 μL injection of 0.025 mg/mL pembrolizumab was injected onto the column, and the binding, elution, and equilibration processes were repeated as described above. This was repeated using pembrolizumab concentrations of 0.05, 0.1, 0.25, 0.5, 0.75, and 1 mg/mL.

The column of Example 3 provided for robust pembrolizumab concentration at low volumes and concentrations. The pembrolizumab eluted from the column with sharp, well-defined peaks (FIG. 3). These data further enabled robust titer analysis with an R² of 0.998.

### Example 5: Streptavidin Leaching Evaluation Method 1

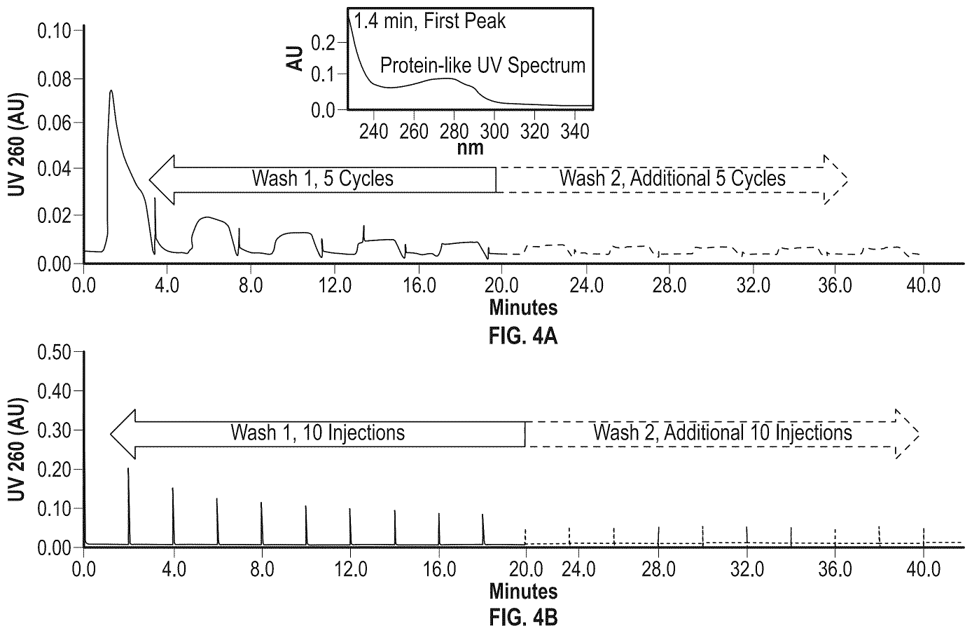

Particles (Example 2, product #2c) were packed in a 2.1×20 mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and equilibrated in running 100 mM sodium phosphate buffer (pH7.4) at 0.4 mL/min. Next, step gradient to 20% acetonitrile with 1% phosphoric acid was applied at 0 min and held till 2 min to wash the column. At 2.01 min the mobile phase switched back to 100 mM sodium phosphate buffer and held till 4 min. This wash cycle was repeated five times (Wash 1 labeled in FIG. 4A). Second wash was applied if needed (Wash 2, dotted line in FIG. 4A). The effluent was monitored using a UV detector (280 nm) (FIG. 4A). The inset UV spectrum in FIG. 4A shows that the leachable peak has typical UV spectra of a protein. The applied washing cycle showed a reduction in leachate.

### Example 6: Streptavidin Leaching Evaluation Method 2

Particles (Example 2, product #2c) were packed in a 2.1×20 mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and equilibrated in running 100 mM sodium phosphate buffer (pH7.4) at 0.4 mL/min. Next, 10 μL injection of 1% phosphoric acid aqueous solution with 20% acetonitrile was injected onto the column. These injections act as short column wash and dislodge non-covalently adsorbed leachate (streptavidin) as a sharp peak. Ten injections were executed (Wash 1, FIG. 4B). The column was then washed with 5 cycles of 1% phosphoric acid aqueous solution with 20% acetonitrile (Method I, described above), and the additional 10 injections of wash solution was repeated (Wash 2, dotted line in FIG. 4B). The effluent was monitored using a UV detector (280 nm). FIG. 4B shows the streptavidin leaching from the column of the particles of Example 2, product #2c. The Method I is an efficient approach to remove the residual (non-covalently bonded) leachates from the sorbent.

Particles (Example 2, products #2c-2 h) were packed in a 2.1×20 mm column hardware and stored in storage buffer (100 mM PBS, pH 7.3, 0.02% sodium azide; at ˜10 mL/g particle). The column was stored at 4° C. until ready for use. The column was connected to a liquid chromatography instrument and equilibrated in running 100 mM sodium phosphate buffer (pH7.4) at 0.4 mL/min. Next, 10 μL injection of 1% phosphoric acid aqueous solution with 20% acetonitrile was injected onto the column and the height of the first (highest) peak was measured at 280 nm (Table 2). The data showed that the change in Step 2 particle washing step and immobilization conditions decreased the height of the first peak of the leaching. It is apparent from FIG. 4A and FIG. 4B that the additional washes performed after sorbent packing can further suppress the leachates to approximately 10-fold lower level.

TABLE 2
The peak height measurements of the columns
tested for streptavidin leaching by Method 2

| Product # | 280 nm Max |
|---|---|
| 2c | 0.52 |
| 2d | 0.22 |
| 2e | 0.04 |
| 2f | 0.32 |
| 2g | 0.32 |
| 2h | 0.36 |

### Example 7: Binding of PD-L1 to a PD-1 Antigen Chromatography Column

The column of Example 3 was used to perform affinity capture of PD-L1 with a PD-1 antigen. The column was connected to a liquid chromatography instrument and equilibrated with a 100 mM sodium phosphate buffer (pH 7.4) for 5-7 minutes at 1 mL min flow rate.

Once equilibrated, 5 μL of 0.1 PD-L1 was injected onto the column in and flowed for 3 minutes at 0.1 mL/min in the presence of a 100 mM sodium phosphate buffer to allow for binding. The column effluent was monitored via a fluorescence detector (280 nm excitation, 350 nm emission). The breakthrough was monitored from 0 minutes to 3 minutes, as shown in FIG. 5A. After the binding phase, an injection elution buffer consisting of 25 μL of 120 mM HCl was flown onto the column, and elution was monitored at a flow rate of 0.1 mL/min. The elution was monitored for 3 minutes (denoted as 0* minutes to 3* minutes in FIG. 5B). A comparative experiment was performed wherein elution was performed using an injection elution buffer consisting of 25 μL of 240 mM phosphoric acid at a flow rate of 0.1 mL/min. The elution was monitored for 3 minutes (denoted as 0** minutes to 3** minutes in FIG. 5C).

The PD-1 antigen column was successfully able to capture and elute PD-L1. Notable, some PD-L1 was present in the breakthrough (peak in FIG. 5A), suggesting the binding was not 100% efficient. Quantification of the PD-L1 peak in FIG. 5A indicates that about 50% of the injected PD-L1 was captured. Further, the retained PD-L1 was eluted from the PD-1 antigen column using both a 120 mM HCl buffer and a 240 mM phosphoric acid buffer.

### Example 8: Binding of PD-L2 to a PD-1 Antigen Chromatography Column

The column of Example 3 was used to perform affinity capture of PD-L2 with a PD-1 antigen. The column was connected to a liquid chromatography instrument and equilibrated with a 100 mM sodium phosphate buffer (pH 7.4) for 5-7 minutes at 1 mL min flow rate.

Once equilibrated, 5 μL of 0.6 mL/min PD-L2 was injected onto the column in and flowed for 3 minutes at 0.1 mL/min in the presence of a 100 mM sodium phosphate buffer to allow for binding. The column effluent was monitored via a fluorescence detector (280 nm excitation, 350 nm emission). The breakthrough was monitored from 0 minutes to 3 minutes, as shown in FIG. 6A. After the binding phase, an injection elution buffer consisting of 25 μL of 120 mM HCl was flown onto the column, and elution was monitored at a flow rate of 0.1 mL/min. The elution was monitored for 3 minutes (denoted as 0* minutes to 3* minutes in FIG. 6B). A comparative experiment was performed wherein elution was performed using an injection elution buffer consisting of 25 μL of 240 mM phosphoric acid at a flow rate of 0.1 mL/min. The elution was monitored for 3 minutes (denoted as 0** minutes to 3** minutes in FIG. 6C).

The PD-1 antigen column was successfully able to capture and elute PD-L2. Notable, some PD-L2 was present in the breakthrough (peak in FIG. 6A), suggesting the binding was not 100% efficient. Quantification of the PD-L2 peak in FIG. 6A indicates that about 50% of the injected PD-L2 was captured. Further, the retained PD-L2 was eluted from the PD-L2 antigen column using both a 120 mM HCl buffer and a 240 mM phosphoric acid buffer.

### Example 9: Preparation and Use of an Antigen Chromatography Column to a Protein Expressed on the Surface of a Cell or Viral Particle

Streptavidin particles as prepared in Example 2 are packed into a column body and connected to a liquid chromatography instrument. The column is purged with 100 mM sodium phosphate buffer (pH 7.4) at 0.1 mL/min. A solution comprising an antigen that is a protein expressed on the surface of a cell is injected onto the column until the column is saturated as monitored by presence of the antigen in the effluent (see Example 3). For example, but not by way of limitation, the antigen may be PD-1, PD-L1, PDL2, angiopoietin, BCMA, CD19, CD20, CD22, CD25, CD30, CD33, CD37, CD38, CD52, CD56, CD123, cMet, DLL, EpCAM, FGF, GD2, HER2, mesothelin, nectin-4, PDGFRalpha, RANKL, SLAMF7, TROP2, VEGF, VEGF-R, Claudin 18.2, DKK1, CD3, CD4, or spike protein, or a fragment of any of said proteins. Additionally or alternatively, the protein may be a protein that is present on the surface of a viral particle. For example, the protein may be a shell protein of adeno-associated virus, such as VP1, VP2, or VP3.

The resultant antigen chromatography column is then used to isolate and purify antibodies that specifically bind to said antigen. For example, if the antigen is nectin-4, the column may be used to isolate and purify antibodies that specifically bind to nectin-4. The antigen chromatography column may also be used to isolate and purify antibodies that are conjugated to additional chemical moieties, such as in the form of an antibody drug conjugate. Additionally or alternatively, the antigen chromatography column may be used to isolate and purify antibodies that are conjugated to lipid nanoparticles.

### Example 10: Preparation and Use of a Mixed Antigen Chromatography Column

Streptavidin particles as prepared in Example 2 are packed into a column body and connected to a liquid chromatography instrument. The column is purged with 100 mM sodium phosphate buffer (pH 7.4) at 0.1 mL/min. A solution comprising a first and a second antigen are injected onto the column until the column is saturated as monitored by presence of the antigen in the effluent (see Example 3). The concentration of the first antigen and the concentration of the second antigen can be adjusted as needed. For example, the first antigen and the second concentration may be at a molar ratio of, for example, 1:1, 2:1, 5:1, 10:1, 1:2, 1:5, or 1:10. The first and second antigen may be a protein expressed on the surface of a cell. For example, but not by way of limitation, the first and second antigen may be selected from the list comprising PD-1, PD-L1, PDL2, angiopoietin, BCMA, CD19, CD20, CD22, CD25, CD30, CD33, CD37, CD38, CD52, CD56, CD123, cMet, DLL, EpCAM, FGF, GD2, HER2, mesothelin, nectin-4, PDGFRalpha, RANKL, SLAMF7, TROP2, VEGF, VEGF-R, Claudin 18.2, DKK1, CD3, CD4, or spike protein, or a fragment of any of said proteins. Additionally or alternatively, the first and/or second antigen may be an antigen that specifically binds to an IgE antibody. Additionally or alternatively, the first and/or second antigen may be a shell protein present on an adeno-associated virus (AAV), such as VP1, VP2, or VP3.

The resultant antigen chromatography column is then used to isolate and purify antibodies that specifically bind to said antigens. The antibodies may be conjugated to additional chemical moieties, such as in the form of an antibody drug conjugate. Additionally or alternatively, the antigen chromatography column may be used to isolate and purify antibodies that are conjugated to lipid nanoparticles.

### Example 11: Preparation and Use of a Ubiquitin Binding Domain Antigen Chromatography Column

Streptavidin particles as prepared in Example 2 are packed into a column body and connected to a liquid chromatography instrument. The column is purged with 100 mM sodium phosphate buffer (pH 7.4) at 0.1 mL/min. A solution comprising an antigen that comprises one or more ubiquitin binding domains is injected onto the column until the column is saturated as monitored by presence of the antigen in the effluent (see Example 3). The antigen may comprise 1, 2, 3, 4, 5, 6, or more ubiquitin binding domains.

The resultant antigen chromatography column is then used to isolate and purify antibodies or proteins that specifically bind to the ubiquitin binding domains. The antibodies or proteins may be conjugated to additional chemical moieties, such as in the form of an antibody drug conjugate.

### Example 12: Preparation and Use of an Antigen Chromatography Column Directed to Biomarkers

Streptavidin particles as prepared in Example 2 are packed into a column body and connected to a liquid chromatography instrument. The column is purged with 100 mM sodium phosphate buffer (pH 7.4) at 0.1 mL/min. A solution comprising an antigen that is a biomarker for a disease or disorder is injected onto the column until the column is saturated as monitored by presence of the antigen in the effluent (see Example 3). The disease or disorder may be, for example, an autoimmune disease or disorder, a neurodegenerative disorder, a hematologic disorder. Additionally or alternatively, the biomarker may be a known allergen that binds to an IgE antibody.

The resultant antigen chromatography column is then used to isolate and purify antibodies that specifically bind to the biomarker. The antibodies may be conjugated to additional chemical moieties, such as in the form of an antibody drug conjugate. Additionally or alternatively, the antibodies may be conjugated to the surface of a lipid nanoparticle.

1. A method of loading a biotinylated antigen to a streptavidin column to form an antigen chromatography column, the method comprising:
a) selecting a streptavidin column having a plurality of particles, each particle comprising:
a nonporous polymer core;
a hydrophilic surface on an outer layer of the nonporous polymer core; and
one or more molecules of streptavidin conjugated to the hydrophilic surface, wherein the streptavidin has a plurality of accessible binding sites,
wherein the particle has an average particle size between 1.0 μm to 10 μm;
b) applying a solution of biotinylated antigens to the streptavidin column such that the biotinylated antigens bind to a portion of the plurality of accessible binding sites of streptavidin to form the antigen chromatography column.

2. The method of claim 1, wherein the biotinylated antigens are applied to the streptavidin column using a high-performance liquid chromatography (HPLC) system, ultra-high performance liquid chromatography (UHPLC) system, or fast protein liquid chromatography (FPLC) system.

3. The method of claim 1, wherein the solution of biotinylated antigens is applied for a time sufficient to bind to at least 50% of the plurality of the accessible binding sites of streptavidin.

4. The method of claim 1, further comprising:
a′) washing the streptavidin column prior to applying the solution of biotinylated antigens.

5. The method of claim 4, wherein washing the streptavidin column comprises applying a wash solvent via a liquid chromatography system, wherein the wash solvent comprises acetonitrile and phosphoric acid or a sodium phosphate buffer.

6. (canceled)

7. (canceled)

8. (canceled)

9. A method for enriching a target antibody, the method comprising:
i) providing the antigen chromatography column resulting from claim 1;
ii) washing the column with a binding buffer;
iii) applying a solution containing the target antibody to the antigen chromatography column; and
iv) washing the column with an elution buffer such that the target antibody is eluted from the column.

10. A method for enriching a target protein, the method comprising:
i) providing the antigen chromatography column resulting from claim 1;
ii) washing the column with a binding buffer;
iii) applying a solution containing the target protein to the antigen chromatography column; and
iv) washing the column with an elution buffer such that the target protein is eluted from the column.

11. (canceled)

12. The method of claim 9, wherein the elution buffer is at least 3 orders of magnitude more acidic than the binding buffer.

13. (canceled)

14. (canceled)

15. The method of claim 9, wherein the target antibody or target protein elutes from the antigen chromatography column with a peak volume of at least two times more than that of the target antibody eluted from a conventional column comprising a plurality of porous particles.

16. The method of claim 9, wherein the target antibody or target protein elutes from the antigen chromatography column in less than 5 minutes.

17. The method of claim 9, further comprising step v) detecting the eluting target antibody or target protein with an ultraviolet spectroscopy detector, a fluorescence spectroscopy detector, and/or a mass spectrometry detector.

18. The method of claim 17, further comprising step vi) washing the antigen chromatography column with an equilibration buffer or the binding buffer and repeating steps ii) through v).

19. (canceled)

20. (canceled)

21. The method of claim 1, wherein the coated hydrophilic surface is selected from the group consisting of: (3-glycidyloxypropyl) trimethoxysilane, (3-glycidyloxypropyl)triethoxysilane, glycidol, glyceroltriglycidyl ether, polyacrylate, and poly(methyl acrylate).

22. The method of claim 1, wherein at least a portion of an interior surface of the antigen chromatography column body is coated with alkylsilyl material, wherein the alkylsilyl material is a hydrophilic, non-ionic layer of polyethylene glycol silane.

23. (canceled)

24. The method of claim 1, wherein the biotinylated antigen is an extracellular protein, a membrane protein, or an intracellular protein.

25. The method of claim 1, wherein the antigen is expressed on the surface of an immune cell or the surface of a cancer cell.

26. (canceled)

27. The method of claim 1, wherein the antigen is PD-1, PD-L1, PDL2, angiopoietin, BCMA, CD19, CD20, CD22, CD25, CD30, CD33, CD37, CD38, CD52, CD56, CD123, LAG-3, cMet, DLL, EpCAM, FGF, GD2, HER2, mesothelin, nectin-4, PDGFRalpha, RANKL, SLAMF7, TROP2, VEGF, VEGF-R, Claudin 18.2, DKK1, CD3, CD4, CTLA-4, clusterin, tau, alpha-synuclein, CNX, MHC, Galectin-9, B7-H5 Vista, spike protein, TNF-α, TIGIT, FcRN, PSMA, meothelin, EpCAM, HER2, or EGFR.

28. The method claim 1, wherein the antigen is a shell protein present in an adeno-associated virus (AAV), wherein the shell protein is VP1, VP2, or VP3.

29. (canceled)

30. The method of claim 1, wherein the antigen comprises a ubiquitin binding domain.

31. (canceled)

32. The method of claim 1, wherein the solution of biotinylated antigens comprises a first biotinylated antigen and a second biotinylated antigen.

33. (canceled)
